(19) United States
(12) Patent Application Publication
(10) Pub. No.: US 2026/0086090 A1
(43) Pub. Date: Mar. 26, 2026

(54) ULTRA-SENSITIVE ANALYTE DETECTION AND QUANTIFICATION USING CATCH AND RELEASE WITH PROXIMITY DETECTION

(71) Applicant: The Children's Medical Center Corporation, Boston, MA (US)

(72) Inventors: Clinton H. Hansen, Brighton, MA (US); Wesley Philip Wong, Cambridge, MA (US)

(73) Assignee: The Children's Medical Center Corporation, Boston, MA (US)

(21) Appl. No.: 19/112,170

(22) PCT Filed: Sep. 14, 2023
(86) PCT No.: PCT/US2023/074276

§ 371 Date: Mar. 14, 2025

Related U.S. Application Data
(60) Provisional application No. 63/407,144, filed on Sep. 15, 2022.

(51) Int. Cl. G01N 33/569; G01N 33/53; G01N 33/543

(52) U.S. Cl. CPC G01N 33/56983; G01N 33/5308; G01N 33/54306; G01N 2333/165

(57) ABSTRACT

Provided herein is a highly sensitive analyte detection method that involves catch and release of a target analyte coupled with a proximity assay. Target analyte is captured using a capture binding partner and is also bound by detection binding partners thereby forming an analyte-biding partner complex. The complex is immobilized, and unbound reactants are physically removed. Thereafter the complex is released and a proximity assay is performed on the complex in order to detects its presence.

### RELATED APPLICATIONS

The application claims the benefit under 35 U.S.C. 119 (e) of U.S. Provisional Application No. 63/407,144 filed Sep. 15, 2022, which is incorporated by reference herein in its entirety.

### FEDERALLY SPONSORED RESEARCH

This invention was made with government support under Grant Numbers GM119537 and CA212827, awarded by the National Institutes of Health. The Government has certain rights in the invention.

### REFERENCE TO AN ELECTRONIC SEQUENCE LISTING

The contents of the electronic sequence listing (C123370259WO00-SEQ-MAT.xml; Size: 17,365 bytes; and Date of Creation: Sep. 11, 2023) is herein incorporated by reference in its entirety.

### BACKGROUND OF INVENTION

Analytes, such as proteins, can be detected in samples by a variety of standard techniques. Some such techniques use “sandwiching” binding partners, one immobilized on a surface to capture the target analyte and one or more conjugated to detection moieties to bind to the immobilized target. The detection moieties are often those that can convert a substrate to a product that can be quantified by fluorescent, chemiluminescent, or colorimetric detection. The amount of target molecule in the sample is then proportional to this signal. One advantage of such techniques is that immobilization allows washing to remove unbound material for a lower background. One disadvantage of such techniques though is that the detection antibodies can nonspecifically bind to the surface, resulting in a high background.

### SUMMARY OF INVENTION

Provided herein is novel method which enables both washing and surface-free detection to enable sensitive detection. The method comprises a “capture and release” step followed by a surface-free proximity assay. Generally, in the first step, a target analyte is captured using a capture (or primary) binding partner, such as a capture antibody, and is also bound by detection binding partners, thereby forming an analyte-binding partner complex. The complex is immobilized, and unbound reactants are physically removed. Thereafter, the complex is released and a proximity assay is performed on the complex in order to detect its presence. The proximity assay is performed on a free-flowing complex and employs detection moieties, such as oligonucleotides, that are conjugated to the detection binding partners.

As described herein, the method results in unexpected and significantly improved detection sensitivity. As explained in the Examples, this method has been used to detect SARS-COV-2 spike protein in saliva samples. The limit of detection was measured to be about 14 molecules per 5 microliters of saliva, corresponding to a concentration of 4.7 attoMolar.

The technique was also used to detect SARS-COV-2 spike protein in saliva samples that were negative using nucleic acid based amplification. In these experiments, using the method of this disclosure, the samples were determined to have about 1000 spike proteins.

This disclosure therefore provides methods for analyte detection that are highly sensitive and that can outperform assays based on nucleic acid amplification.

Thus, one aspect of this disclosure provides a method of detecting a target analyte, comprising
combining a capture binding partner and a detection binding partner with a sample, under conditions sufficient for the capture binding partner and detection binding partner, simultaneously or consecutively, in any order) to bind to a target analyte present in the sample, thereby forming an analyte-binding partner complex,
immobilizing the analyte-binding partner complex to a solid support,
removing unbound capture binding partner and detection binding partner,
releasing the analyte-binding partner complex (from the solid support), optionally transferring the released analyte-binding partner complex to a second container, and
performing a proximity assay on the released analyte-binding partner complex in solution, thereby detecting the presence of the analyte-binding partner complex, and thus the analyte.

Another aspect of this disclosure provides a method of measuring analyte complex size, comprising
combining, in the presence and in the absence of an unlabeled capture binding partner, a labeled capture binding partner and a detection binding partner with a sample, under conditions sufficient for the labeled capture binding partner and the detection binding partner, to bind to a target analyte (or analyte complex) present in the sample, thereby forming an analyte-binding partner complex,
immobilizing the analyte-binding partner complex to a solid support,
removing unbound capture binding partner and detection binding partner,
releasing the analyte-binding partner complex (from the solid support), optionally transferring the released analyte-binding partner complex to a second container, and
performing a proximity assay on the released analyte-binding partner complex in solution, thereby detecting the presence of the analyte-binding partner complex, and thus the analyte or analyte complex,
wherein the difference in signal from the proximity assay between the method performed in the presence of the unlabeled capture binding partner and the method performed in the absence of the unlabeled capture binding partner is indicative of analyte complex size. In this aspect, an analyte complex refers to an analyte-comprising complex that exists in the sample prior to performing any of the methods provided herein, and is not to be confused with the analyte-binding partner complex described herein which is formed upon binding of the analyte (or the analyte complex) with one or more capture binding partners and the detection binding partners.

Another aspect of this disclosure provides a method of detecting a target analyte, comprising
forming an analyte-binding partner complex comprising a target analyte bound to a capture binding partner and two or more (e.g., 3 or more) detection binding partners, wherein the capture binding partner is immobilized to a solid support before or after binding to the target analyte,
separating the analyte-binding partner complex from unbound capture binding partner and/or unbound detection binding partner,
releasing immobilized analyte-binding partner complex from the solid support, and optionally transferring the released analyte-binding partner complex to a second container,
performing a surface-free proximity assay on the released analyte-binding partner complex, thereby detecting the presence of the analyte-binding partner complex and thus the analyte.

Various embodiments relate to each of the foregoing aspects and these will be recited below for brevity.

In some embodiments, the target analyte is a protein or a peptide.

In some embodiments, the capture binding partner is amino acid or nucleic acid in nature.

In some embodiments, the capture binding partner is an antibody or antigen-binding antibody fragment, a nanobody, a single chain antibody, or an aptamer.

In some embodiments, the detection binding partner comprises an analyte-specific binding partner conjugated to a detection moiety, such as an oligonucleotide that acts as a substrate for a proximity assay.

In some embodiments, the analyte-specific binding partner is amino acid or nucleic acid in nature.

In some embodiments, the analyte-specific binding partner is an antibody or antigen-binding antibody fragment, a nanobody, a single chain antibody, or an aptamer.

In some embodiments, the detection moiety is an enzyme substrate, an enzyme, a FRET donor, or a FRET acceptor.

In some embodiments, the detection moiety is an oligonucleotide, such as an oligonucleotide that acts as a substrate for a proximity assay.

In some embodiments, the sample is a bodily sample, optionally a saliva sample, a sputum sample, a nose or nasopharyngeal swab sample.

In some embodiments, the target analyte is produced by a pathogen, optionally a virus, a bacterium, or a fungus. In some embodiments, the target analyte is present on or in a pathogen, such as a virus, a bacterium or a fungus.

In some embodiments, the target analyte is SARS-COV-2 spike protein.

In some embodiments, the target analyte is present in the sample at a concentration of about or at least 5 attoMolar (aM), or about or at least 10 aM, or about or at least 15 aM, or about or at least 20 aM, or in the range of about 5 aM to about 1000 aM, or in the range of about 5 aM to about 500 aM, or in the range of about 5 aM to about 200 aM, or in the range of about 5 aM to about 100 aM, or in the range of about 5 aM to about 50 aM.

In some embodiments, the solid support is a surface of a well or tube or bead.

In some embodiments, the capture binding partner is conjugated to a first oligonucleotide, the solid support is conjugated to a second oligonucleotide, the capture binding partner is immobilized to the solid support by hybridization of the first and second oligonucleotides to each other, and optionally the analyte-binding partner complex is released by nucleic acid strand displacement in the presence of a release oligonucleotide. The release oligonucleotide may be complementary to the first oligonucleotide, or the release oligonucleotide may be complementary to the second oligonucleotide.

In some embodiments, the capture binding partner is immobilized to the solid support by a ultraviolet (UV) cleavable linker, and the analyte-binding partner complex is released by exposure to ultraviolet (UV) light.

In some embodiments, the capture binding partner is immobilized to the solid support by a nucleic acid linker, and the analyte-binding partner complex is released by contact (and subsequent cleavage) with a restriction enzyme.

In some embodiments, during or following the release of the analyte-binding partner complex the solid support is exposed to a quenching agent to prevent re-association of the released analyte-binding partner complex.

In some embodiments, the solid support is a removable component, such as a bead or a strip, and following the release of the analyte-binding partner complex such solid support is removed from the container or vessel rather than transferring the complex to a new container or vessel.

In some embodiments, the detection binding partner is two detection binding partners, and each detection binding partner is conjugated to an oligonucleotide that is a substrate in a proximity assay. In some embodiments, the detection binding partner is three detection binding partners, and each detection binding partner is conjugated to an oligonucleotide that is a substrate in a proximity assay. The analyte or sample may be combined with the capture binding partner and the detection binding partners in any order, including simultaneously or sequentially.

In some embodiments, the proximity assay is a proximity ligation assay.

In some embodiments, the proximity assay is a proximity extension assay.

These and other aspects and embodiments of this disclosure will be described in greater detail herein.

### BRIEF DESCRIPTION OF DRAWINGS

The accompanying drawings are not intended to be drawn to scale. Certain of the accompanying drawings may be in color and these may be accessed through the file wrapper at the United States Patent and Trademark Office.

FIG. 1. Schematic for the general catch and release scheme described by this disclosure. The target analytes are immobilized onto a surface via the capture binding partner such as an antibody. The target analytes are also bound to detection binding partners such as antibodies. As illustrated, one capture (or primary) antibody and two detection antibodies are bound to a target analyte. After washing, the capture antibody is released and transferred to another vessel for detection through a proximity detection technique such as, but not limited to, a proximity ligation assay.

FIG. 2. Dose response curve of catch and release detection of SARS-COV-2 spike protein S1 in saliva. Detected number of S1 protein counts as a function of actual number of S1 proteins spiked into 5 μl of human saliva using catch and release coupled with a proximity ligation assay. The detected counts were determined from the cycle threshold (Ct) values from qPCR using a generated standard curve from an oligonucleotide corresponding to the proximity ligation product. Error bars are the standard deviations over 4 replicates.

FIG. 3. Catch and release detection of S1 protein counts using a proximity ligation assay compared to SalivaDirect in patients. Matched SalivaDirect qPCR and catch and release results 5 μl of patient saliva from COVID− and COVID+ patients, with COVID+ patients classified from matched nasopharyngeal samples. The dashed line is the determined cutoff between classification of COVID+ and COVID− from catch and release (CaR) given by the background plus three standard deviations of the background.

FIGS. 4A and 4B. Complex size detection using catch and release.

FIG. 4A. Inferred complex size when using antibodies against the S1 domain of the SARS-COV-2 spike protein. The ratio of the signal in the presence of 20× unlabeled (i.e., not capable of being immobilized) capture antibody is given for samples of the S1 portion of the spike trimer, the extracellular domain (ECD) of the spike trimer, a SARS-COV-2 pseudovirus, and a COVID+ patient. The inferred complex size given above each bar was calculated as log (1-ratio)/log (blocking antibody proportion).

FIG. 4B. Inferred complex size when using antibodies against the S2 domain of the SARS-COV-2 spike protein. The ratio of the signal in the presence of 10×, 30×, or 90× unlabeled capture antibody is given for samples of the ECD of the spike trimer and a SARS-CoV-2 pseudovirus. The inferred complex size given above each bar was calculated as log (1-ratio)/log (blocking antibody proportion), using 10× blocking antibody for the trimer and 30× blocking antibody for the pseudovirus.

### DETAILED DESCRIPTION OF CERTAIN EMBODIMENTS

Provided herein is novel method that combines a “catch and release” step coupled with a “surface-free” detection proximity assay, and unexpectedly results in higher sensitivity than assays that rely on nucleic acid amplification.

Generally, in the first step, a target analyte is bound to a capture (or primary) binding partner, such as a capture antibody, and to detection binding partners. The result of this first step is the formation of an analyte-binding partner complex. The capture binding partner may be immobilized before or after binding to the target analyte, and accordingly the complex is immobilized to a solid support. Once the complex is immobilized, unbound components, such as unbound capture and detection binding partners, may be washed away. Once the unbound components are physically separated from the immobilized complex, the complex is released and transferred to a second container. The immobilization and thus release of the capture binding partner can be achieved using a variety of techniques including but not limited to a UV cleavable linker or nucleic acid strand displacement, with or without quenching to prevent the re-association of one or more reaction components (such as short oligonucleotides or Twin Strep-Tag). In the case of Twin-Strep-Tag, for example, the addition of biotin serves to quench the Tag on the capture binding partner in order to prevent it from reassociating specifically with the surface. These techniques will cause the specific release of the analyte-binding partner complex while reaction components that are non-specifically bound to the solid support will remain attached.

In the second step, a proximity assay is performed on the released analyte-binding partner complex. The proximity assay generates a read-out provided that two (or more) detection binding partners are sufficiently close to each other to allow interaction of oligonucleotides attached to each. The oligonucleotides may be ligated to each other to form a new nucleic acid or they hybridize to each other and thereby generate a template from which nucleic acids may be synthesized.

In one aspect, the method comprises
(1) combining capture (or primary) binding partners and detection binding partners with a sample, under conditions sufficient for a capture binding partner and two or more detection binding partners to bind to a target analyte present in the sample, thereby forming an analyte-binding partner complex
(2) immobilizing the analyte-binding partner complex to a solid support,
(3) physically separating the analyte-binding partner complex from unbound capture binding partners and detection binding partners,
(4) releasing the analyte-binding partner complex from the solid support,
(5) optionally transferring the released analyte-binding partner complex to a second container, and
(6) performing a proximity assay on the released analyte-binding partner complex in solution, thereby detecting the presence of the analyte-binding partner complex.

In another aspect, the method comprises
(1) forming an analyte-binding partner complex comprising a target analyte bound to a capture binding partner and two or more detection binding partners, wherein the capture binding partner is immobilized to a solid support before or after binding to the target analyte,
(2) separating the analyte-binding partner complex from unbound capture binding partners and unbound detection binding partners,
(3) releasing immobilized analyte-binding partner complex,
(4) optionally transferring the released analyte-binding partner complex to a second container, and
(5) performing a surface-free proximity assay on the released analyte-binding partner complex, thereby detecting the presence of the analyte-binding partner complex.

In some embodiments, the immobilization occurs on a removable surface in a vessel or container, such as but not limited to a strip. As another example, the surface may be a bead surface and such beads may be physically separated from the remainder of the reaction (or reaction solution). If the beads are magnetic, then they may be physically separated from the remainder of the reaction by applying a magnetic force. Alternatively, the catch and release step may be performed in a column onto which the capture binding partner (or the analyte-binding partner complex) may be bound, and then subsequently released. In still other embodiments, the analyte-binding partner complex may be released from a surface of a vessel and then transferred, manually or automatically, to another chamber, vessel, container, or the like.

The proximity assay is referred to as occurring “in solution” or “surface-free”, intending that the analyte-binding partner complex is free in solution and not immobilized and/or in the absence of the surface upon which the catch and release step was performed. Thus, although the proximity assay will be performed in a container, vessel, chamber or the like, all of which will have their own inherent surfaces, the analyte-binding partner complex does not have to be bound, and in some instances will not be bound, to any such surface in order for the proximity assay to be performed.

### Catch and Release Step

The target analyte or sample containing or suspected of containing the target analyte is combined with capture binding partners and detection binding partners. At a minimum, a target analyte should be bound to a capture binding partner and two detection binding partners. The capture and detection binding partners may bind to identical epitopes that are repeated on the target analyte, or they may bind to different epitopes on the target, provided that the capture and detection binding partners can bind to the target analyte simultaneously. In some embodiments, the detection binding partners may bind to an identical repeated epitope. In some embodiments, the proximity assay may require that three detection binding partners be bound to the target analyte. The complex comprising the target analyte bound to the capture binding partner and the detection binding partners is referred to as the analyte-binding partner complex.

The detection binding partners are themselves conjugated to detection moieties. The detection moieties may be oligonucleotides that serve as substrates for the proximity assay. The detection oligonucleotides will typically be different from each other. Equimolar amounts of detection binding partners are typically used, particularly where the binding affinities of the detection binding partners for the analyte are about the same.

The order of addition of the various components to the sample may vary. The capture binding partner may be added first or the capture and one or more of the detection binding partners may be added together. In the working examples, the sample was combined with the capture binding partner (e.g., antibody) and a first detection binding partner (e.g., antibody A). The complex so formed was then immobilized on the solid support, washed and then incubating with a second detection binding partner (e.g., antibody B). Other orders can be used. For example, the sample may be combined with the capture binding partner and the detection binding partners before immobilization on the solid support. As another example, the sample may be combined with the capture binding partner, then the resultant complex may be immobilized and then washed, and then incubated with the detection binding partners, simultaneously or sequentially. In yet another example, an immobilized capture binding partner may be contacted with the sample, and the sample may be contacted with one or more of the detection binding partners, simultaneously or sequentially. In still other variations, the sample may be contacted with the detection binding partner and then contacted with the capture binding partner which may already be immobilized.

Accordingly, the capture binding partner is immobilized to a solid support before, during or after formation of the analyte-binding partner complex. Immobilization of the capture binding partner may be achieved using a variety of ways. As an example, the capture binding partner may be immobilized using a linker bound to the solid support on one end and to the capture binding partner on the other end. The linker may be cleaved upon exposure or contact with an agent such as an enzyme or a chemical or with energy such as ultraviolet (UV) light. Thus, one type of suitable linker is a UV cleavable linker. Another type of suitable linker is an oligonucleotide that can be cleaved by a restriction enzyme. These linkers may be bound to the solid support using a biotin-avidin (or biotin-streptavidin). They may be bound to the capture binding partner through covalent attachment. The working examples provided herein use a biotinylated oligonucleotide immobilized on streptavidin-coated magnetic beads.

As another example, the solid support may be conjugated to a first oligonucleotide and the capture binding partner may be conjugated to a second oligonucleotide that is at least partially complementary to the first oligonucleotide. The capture binding partner (and accordingly the analyte-binding partner complex) may be released from the solid support through a process of strand displacement which involves addition of a third oligonucleotide that displaces the first oligonucleotide from the second oligonucleotide. The third oligonucleotide, which may be referred to herein as a release oligonucleotide, generally has greater affinity for the second oligonucleotide than does the first oligonucleotide. Strand displacement is described in Zhang et al., 2011.

The catch and release step may also involve quenching reactive sites on the solid support to prevent re-association of the capture binding partner or association of the detection binding partners. As an example, if the catch and release step employs a strand displacement mechanism, then upon release of the analyte-binding partner complex, a quenching oligonucleotide may be added to hybridize with the first oligonucleotide.

Alternatively, the release oligonucleotide may have greater affinity for the first oligonucleotide than the second oligonucleotide and the quenching oligonucleotide may be complementary to the second oligonucleotide.

Quenching can also be performed using agents that compete for binding to biotin or streptavidin or streptavidin derivates such as Strep-Tactin or Strep-Tactin-XT, including Twin-Strep-tag.

The analyte-binding partner complex is released and then optionally transferred to a new container (or vessel) in order to further reduce interfering non-specific binding to the solid support.

The catch and release step can also be combined with other additional steps to reduce background. These can include, but are not limited to, a depletion step in which the released solution is added to a well or beads with immobilized binding entities that can remove unbound detection binding partner or unbound detection oligonucleotides from the solution, as an intermediate step and before transferring the solution to another container in which the proximity assay or detection step is performed. In another embodiment, the detection step may be carried out by transferring the released solution to a well or put in contact with beads having immobilized binding entities that can bind to the analyte-binding partner complex at an additional site and thereby immobilize the complex and allow further washing followed by detection using the proximity assay. This latter embodiment may further comprise an additional release and transfer step to even further reduce the background. Additionally, the catch and release step can be performed multiple times (once, twice, or more times) with 2 or more capture binding partners caught and released in sequence, before a final detection step using a proximity assay. Additionally, another variation could include an extra incubation step for the released and transferred solution to disassociate any non-specifically bound detection binding partners or oligonucleotides, potentially through the addition of compounds that can bind to free binding partners or oligonucleotides to keep them apart. Another variation could include binding each detection binding partner to the target analyte or the target complex in succession in different incubation steps after capturing the target in order to prevent high concentrations of detection binding partners or oligonucleotides from encountering each other in solution.

In another variation, catch and release can be used to reduce the volume necessary for performing a qPCR, such as part of a proximity assay or other detection step, from a large sample volume, as the release volume can be controlled.

### Proximity Assay or Detection Step

The catch and release step is combined with a proximity assay that detects the analyte-binding partner complex. The coupling of these two steps serves to simultaneously enable both extremely low background and extremely high sensitivity.

Typically, a proximity assay detects an analyte (or in this case an analyte-containing complex) by detecting two or more probes (e.g., oligonucleotides) that are in close proximity when they simultaneously bind to a single target. In the context of this disclosure, the probes are the oligonucleotides conjugated to detection binding partners, and when two (or more) detection binding partners are bound to the target analyte the probes are brought into sufficiently close proximity to be detected. The proximity assay may take different forms, including but not limited to a proximity ligation assay (PLA) and a proximity extension assay (PEA).

In a proximity ligation assay (PLA), simultaneous binding of two antibody-oligonucleotide conjugates to a single target allows the two oligonucleotides in close proximity to be ligated together to form a single DNA strand (Fredriksson et al., 2002). The resultant DNA product can then be detected through any quantitative method that can detect a specific DNA sequence, such as quantitative PCR (qPCR, real-time PCR or RT-PCR). In the absence of target analyte, the probes are not in close proximity, and no new DNA products are formed.

In a proximity extension assay (PEA), two oligonucleotides in close proximity hybridize to each other, and DNA polymerase extends one of the hybridized oligonucleotides, to form a new DNA strand (Lundberg et al. 2011a). As with PLA, the end product of PEA may be detected using qPCR or by sequencing or through the activity of a split enzyme such as split horseradish peroxidase (Martell et al., 2016).

Variations on these assays include a 3-body assay that requires the presence of three oligonucleotides in order to form a new DNA strand to be detected (Schallmeiner et al., 2007). The requirement for more proximity oligonucleotides for detection will further reduce the background over just using a single oligonucleotide (or probe) for detection.

In another variation, the release oligonucleotide could also include a detection oligonucleotide to be used in the proximity assay, potentially as a separate entity from the cleavable linker, as a portion of the cleavable linker, or that forms upon cleavage of the linker. This could allow the use of a 3-body proximity assay when 2 other proximity probes (e.g., oligonucleotides) are used, or a 2-body assay in which only one other proximity probe (e.g., oligonucleotide) is used.

Coupling the proximity assay with a catch and release step, as described herein, provides reduced background compared to a proximity assay used alone. This may be in part due to the ability to physically separate excess sample and unbound binding partners from the immobilized analyte-binding partner. Additionally, the proximity assay is performed on an analyte-binding partner complex that is in solution (i.e., not immobilized or in the absence of a capture surface that might otherwise create background by non-specifically binding two detection binding partners in close proximity.

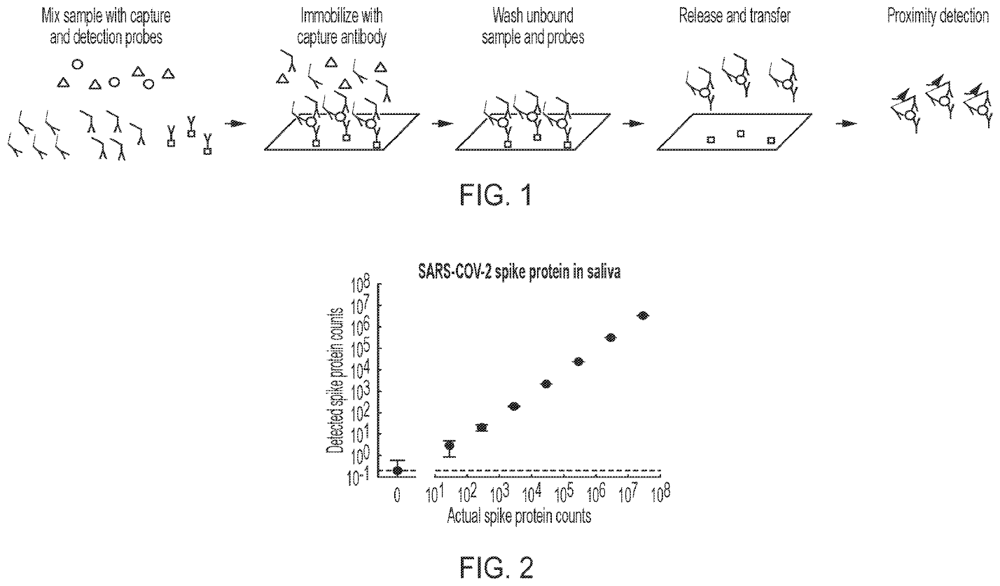

FIG. 1 provides a schematic demonstrating capture and release coupled with proximity detection. Since this method reduces background significantly, it enables close to single molecule protein detection in complex fluids, such as saliva or serum.

In a variation of the foregoing proximity assay, the analyte-binding partner complex may be detected through other means. For example, this disclosure contemplates that the detection moiety may be detected through fluorescent, chemiluminescent, or colorimetric signal. In one embodiment, this may be accomplished by conjugating one detection binding partner to an enzyme and conjugating another detection binding partner to the enzyme's substrate. The enzyme may be horse-radish peroxidase or alkaline phosphatase enzyme, without limitation. When the two detection moieties are brought into close proximity, so are the enzyme and substrate, resulting in the detection of the product converted from the substrate by the enzyme. Other detection mechanisms are also contemplated including Fluorescence Resonance Energy Transfer (FRET), a split enzyme or fluorescent protein, or colocalization of two different bead type.

All readouts can either be done in bulk or using a digital readout of microwells, such as digital qPCR (Vogelstein & Kinzler 1999) or digital ELISA (Rissin et al. 2010). For example, the method may be perform qPCR in multiple wells or microwells for a single sample for a digital qPCR readout, or dividing the released solution for a split enzyme into multiple wells or microwells for a digital enzyme assay.

In a variation of the foregoing, “blocking” partners such as “blocking” oligonucleotides that bind to the first and/or second oligonucleotides conjugated to the detection binding partners can be used, including at a high concentration. These blocking partners can be used to bind to, and thereby quench, first and/or second oligonucleotides that are not bound to each other. The blocking partners can be used at a concentration and/or can have a binding affinity that allows them to bind to “free” first and/or second oligonucleotides, but not significantly interfere with pre-existing interactions between bound first and second oligonucleotides. This is because the local concentration and affinity for bound detection oligonucleotides to each other is much higher than free detection oligonucleotides. Additionally, the blocking partners are designed so that when a first or second oligonucleotide binds to or ligates with a blocking partner instead of with the second or first oligonucleotide, no signal results. Therefore blocking partners further decrease the background by decreasing the extraneous signal from free detection moieties, while not significantly decreasing signal from bound detection moieties in close proximity.

As an example, in the proximity ligation assay described above, suitable blocking partners can be short oligonucleotides that are identical to the ends of the PLA oligonucleotide sequences, but which include deoxyUridine bases that are cleaved upon treatment with uracil-N-glycosylase (UNG), so the resultant product is not detected during qPCR.

Alternatively, blocking partners may be short oligonucleotides that are partially identical to the ends of the PLA oligonucleotide sequences, but may include additional non-identical bases and/or a modified base at the end to prevent further ligations or extension by a polymerase. Such a modified base may be an inverted T, which prevents ligation, and for each blocking partner the 3-15 bases on either the 3′ end or phosphorylated 5′ end are identical to the detection probes and an additional 3-10 bases are non-identical. When the detection probes are ligated to the “blocking” partners, they are not detected through qPCR since the resulting sequence does not include regions for both forward and reverse primers to bind, and the ends cannot be efficiently extended. For a split enzyme assay, these blocking partners may include antibodies or nanobodies that bind to the interaction surface.

### Measuring Complex Size

In addition to sensitive single analyte detection, the catch and release assay can be used to measure the size of a complex that comprises an analyte (referred to herein as an analyte complex). This is accomplished by measuring the signal change when adding unlabeled, blocking binding partner, such as a blocking antibody, that competes with the capture binding partner, such as capture antibody, for binding to the target analyte (or analyte complex) in solution. The unlabeled blocking binding partner is able to bind to the target analyte but is not capable of being specifically immobilized onto the solid support or surface. If the target analyte can only bind a single capture antibody, as an example, then each target analyte binds to either the capture antibody or the competing blocking antibody. If the target analyte binds to the capture antibody, then the assay will continue as described herein and signal will be ultimately detected from the proximity assay. If the target analyte binds to the blocking antibody and is therefore in solution, it will be washed away and lost. The total signal counts will be lower in that event and may underrepresent the total analyte content of a sample.

The blocking antibody may be used to detect size of an analyte complex that is capable of binding more than one capture antibody at a time. In instances in which the target analyte, or analyte complex, is able to bind multiple capture antibodies simultaneously, the decrease in signal output is lower than if each target analyte (or analyte complex) can only bind a single capture antibody.

The complex size can be calculated from the ratio of the signal generated with blocking antibody to signal generated without blocking antibody and the proportion of unlabeled, blocking antibody, specifically as log (1-signal ratio)/log (blocking antibody proportion).

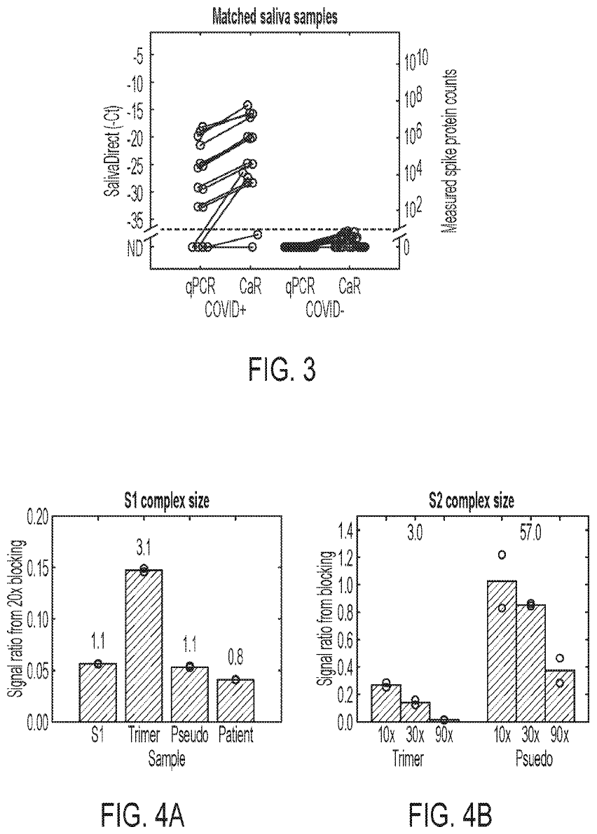

The working examples describe experiments directed to the S1 and S2 portions of the SARS-COV-2 spike protein (FIG. 4). Using the same detection antibodies as in FIGS. 2 and 3, the complex size was measured for the S1 portion of the spike-trimer (FIG. 4A). As expected, for a target consisting of the S1 portion of the spike protein, the complex size is ˜1, while for a stabilized version of the ECD of the full spike-trimer, the complex size is ˜3. For both pseudovirus and COVID+ patient samples, the measured complex size is ˜1. This is expected as the native spike-trimer is unstable and the S1 portion detaches from the rest of the virus to form monomers.

Complex size was also measured using antibodies against the S2 portion of the spike-trimer (FIG. 4B). For the trimer protein, an S2 complex size of ˜3 was measured as expected, and for the pseudovirus sample an S2 complex size of ˜57 was measured, reflecting that each pseudoviral particle contains numerous spike-proteins.

This method may be used to measure complex size for aggregated proteins, such as those involved in neurodegeneration. Other analyte complexes that may be measured using this method include vesicles, cells, cell fragments, viruses, and the like.

This method can be used to measure complex size using any technique that uses an immobilized capture antibody. The highly-sensitive catch and release method of this disclosure allows complex size determination for samples with very few target analytes (e.g., very few protein particles).

### Analytes

Target analytes to be detected using the methods provided herein may be proteins, peptides, nucleic acids, small molecules, lipids, carbohydrates, and the like. The target analyte must be capable of being bound by at least three binding partners (i.e., the capture binding partner, and two or more detection binding partners) at the same time. Therefore, while the working examples provided herein and some of the embodiments provided herein may refer to analytes that are proteins, it is to be understood that the method is not so limited.

The analytes may be of human origin or they may be of pathogen origin. For example, the analyte may be a cancer antigen. Alternatively, it may be a virus, a bacterium, a fungus, or a portion thereof such as a viral protein, a bacterial protein, a fungal protein, etc. The working examples demonstrate the detection of spike protein from SARS-COV-2.

### Samples

The disclosure contemplates that samples of various origins will be tested for the presence of target analytes. The samples may be known to contain the target analyte, or it may be suspected of containing the target analyte, or it may not be known a priori whether the sample contains the target analyte. The method therefore may be used as a first line diagnostic or as a confirmatory assay. In this regard, the method may be used to detect the presence of a pathogen such as a virus or measure the degree of pathogen load such as viral load.

The sample may be a bodily fluid such as but not limited to a saliva sample, a nasopharyngeal sample, a sputum sample, a serum sample, a blood sample, a cerebrospinal fluid sample, a urine sample, etc. The sample may be unmanipulated or it may be manipulated ahead of performing the methods of this disclosure. For example, the sample may be purified exosomes/extracellular vesicles, lysed exosomes/extracellular vesicles, cell lysate, or cell supernatant.

Alternatively, the sample may be an environmental sample such as a water sample, an air sample, a soil sample, a food sample, or a waste water treatment sample, although it is not so limited.

### Capture and Detection Binding Partners

The binding partners may be any moiety that binds with specificity to a target analyte, and that can be conjugated to an oligonucleotide or other linker type. The working examples and various embodiments herein refer to binding partners that are antibodies but it is to be understood that other binding partners are also contemplated including but not limited to aptamers, small molecules, receptors or ligands of the target analyte, and the like. The binding partners may be antibodies or antigen-binding antibody fragments such as single chain antibodies, Fab′ fragments, F(ab′)2 fragments, bispecific Fab dimers (Fab2), trispecific Fab trimers (Fab3), Fv, single chain Fv proteins (“scFv”), bis-scFv, (scFv) 2, minibodies, diabodies, triabodies, tetrabodies, disulfide stabilized Fv proteins (“dsFv”), and single-domain antibodies (sdAb, Nanobody).

### Immobilization Surfaces

Capture binding partners and/or analyte-binding partner complexes may be immobilized on solid supports. Solid supports used for immobilization can be any inert support or carrier that is essentially water insoluble and useful in immunometric assays, including supports in the form of, e.g., surfaces, particles, porous matrices, etc. Examples of commonly used supports include small sheets, Sephadex, polyvinyl chloride, plastic beads, and assays plates or test tubes manufactured from polyethylene, polypropylene, polystyrene, and the like including 96-well microtiter plates, as well as particulate materials such as filter paper, agarose, cross-linked dextran, and other polysaccharides.

In some embodiments, the solid supports are wells, beads or a column, optionally coated with a chemical group or agent that can react with a linker or directly with the capture binding partner either covalently or noncovalently. Migration time under flow or electrophoresis through a column or other substrate comprising affinity entities to the target can also be used for purification.

In the working examples provided herein, similar buffers were used for washes and release. In other variations, the wash buffer can be optimized to reduce non-specific binding, and this may include as an example adding or increasing the concentration of a surfactant (such as Tween 20) and/or adding or increasing the concentration of a salt (such as NaCl). Conversely, the release buffer can be optimized to prevent the release of non-specifically bound detection moieties during the release step, and this may include decreasing the concentration of surfactant(s) and/or salts, or not using them at all. Alternatively the release buffer can be optimized for downstream detection steps, such as a proximity ligation.

Additionally, wash and/or release conditions may be varied to achieve lower backgrounds. For example, the release steps may be performed under conditions that favor non-specific binding of detection binding partners (or other components) to the solid support. The release steps may be performed at room temperature or lower or with buffers that encourage the non-specific binding but do not interfere with the release mechanism used to disassociate the capture binding partner from the solid support. One advantage of the methods provided herein is that the association and disassociation of capture binding partners to the solid support is specific and can be modulated using parameters that have little or no impact on those components that are non-specifically associated with the solid support. In this way, the complexes of interest may be released from the solid support, leaving behind the non-specifically bound components, which might otherwise create background.

### Multiplexing

The methods disclosed herein can also be used in multiplexing embodiments in which catch and release is used to detect a plurality of analytes from a single sample. Multiplexed methods may involve the creation of distinct nucleic acid molecules that are different for each target analyte by coupling detection binding partners to different sequences, either for direct detection or detection using PLA or PEA. These distinct sequences can then be quantified through multiplex qPCR with different TaqMan probes that target each distinct nucleic acid molecule or high-throughput sequencing.

The catch and release method is also contemplated for use in screening for 3-body interactions using a nucleotide-encoded library, such as a DNA-encoded small molecule library or mRNA display library.

### Kits and Devices

Also provided herein is a kit. Kits comprising one or more components useful for performing the methods described herein can include but are not limited to, any necessary components, reagents, or materials necessary to perform methods described herein, and/or instructions for performing the methods described herein. The kit can optionally include any additional washing agents, incubation containers, solid support surfaces, and the like for carrying out the methods described herein.

In one embodiment, the kit may comprise 1, 2 or more detection binding partners conjugated to detection oligonucleotides, a solid support (e.g., a well or a multiwell plate) having immobilized to its surface capture binding partners through a mechanism that allows quick release, and releasing agent, such as a release oligonucleotide, that releases the capturing antibody from the surface and into solution. The kit may include oligonucleotides that can be conjugated to user-provided analyte-specific binding partners such as analyte-specific antibodies. Instructions for conjugation of binding partners to detection oligonucleotides may also be provided. Instructions for quantification of samples including complex samples may also be provided.

Also provided herein is a device that performs, including automatically performs, some or all the actions required for detection, as described herein. The device may include a robotic pipetting machine, a liquid handling robot, and/or magnetic bead handler robot to add a sample, wash the sample, add the releasing agent, and/or transfer the released target analyte or analyte-binding partner complex to a new container or vessel (e.g., a plate or tube). The device may be a microfluidic device that can add the sample, wash the sample, add the releasing agent, and/or transfer the released target analyte or analyte-binding partner complex to a new well in the device or to an external plate or tube.

Some of the embodiments, advantages, features, and uses of the technology disclosed herein will be more fully understood from the Examples below. The Examples are intended to illustrate some of the benefits of the present disclosure and to describe particular embodiments but are not intended to exemplify the full scope of the disclosure and, accordingly, do not limit the scope of the disclosure.

### EXAMPLES

### Materials and Methods

Antibodies purchased from Genscript were coupled to the azide-modified oligonucleotides given in Table 1 as previously described (Hansen et al. 2017). Briefly, we mixed the antibody, the azide-modified oligonucleotide and a bifunctional DBCO-peg4-NHS linker, and incubated for 4 hours. Then we purified coupled antibodies using a BluePippin instrument. For this assay we used DNA strand-displacement to release the capture antibody from the surface. Specifically, the capture antibody is tagged with a 20mer 3′-azide-modified oligonucleotide (Tag). This 20-mer is partially complimentary to a 13-mer capture oligonucleotide with a 3′ biotin modification (Capture). To release the capture antibody from the capture oligonucleotide, a 20mer release oligonucleotide (Release) is added that is fully complementary to the 20mer oligonucleotide that is conjugated to the capture antibody. To reduce any extraneous signal from qPCR, any DNA sequences that are not detection probes can contain deoxyUridine bases that cleave upon treatment with the enzyme uracil-N-deglycosylase (UNG) before qPCR to reduce any extraneous signal. These include, but are not limited to the splint, the release oligonucleotide, tag oligonucleotide, and any additional blocking partners.

The beads with biotinylated capture oligonucleotides were prepared by washing a 1:10 dilution of dynabeads 4 times with 1×PBS, the incubating for 1 hour with 200 nM of capture oligonucleotide. Then the beads were washed 3 times with 1×PBS, incubated 2 times for 15 minutes in casein blocking solution (Thermo Fisher), and then washed 3 times with 1×TBST (25 mM Tris, 0.15M NaCl, 0.05% Tween-20, pH 7.5).

The 5 μl saliva sample was mixed with the capture antibody, detection antibody A. and Halt Protease Inhibitor Cocktail (and blocking antibody if specified) and 1×TBST buffer to a final volume of 25 μl for 1 hour. Then the solution was added to the prepared beads and incubated for 30 minutes. Then the beads were washed 2 times with 1×TBST and incubated with detection antibody B for 1 hour. Then the beads were washed 3 times with 1×TBST for 10 minutes each. The antibody-target protein complexes were then released by incubating with 200 nM of release oligonucleotide in 1×TBST with 5 mM MgCl2 for 10 minutes. The resulting solution was transferred to 8-well strips (Axygen PCR-0208-CP-C) and 2 times volume of 1.25 μM splint oligonucleotide in ligation buffer (50 mM KCl, 1.5 mM MgCl2, 10 mM HEPES pH 7.5, 1 mM ATP) was added and incubated for 15 minutes at room temperature. Next the same 2 times volume of T4 DNA ligase (New England Biolabs) diluted to 8000 units/ml in 1×TBST was added and incubated for another 15 minutes. Then the ligase was inactivated at 65° C. for 10 minutes. The resulting solution assayed using a TaqMan qPCR assay.

### Results

We demonstrated our catch and release detection technique through detection of the SARS-COV-2 spike protein in saliva. In this implementation we used 3 antibodies against the S1 portion of the spike-trimer (Genscript), one as a capture antibody and 2 as detection antibodies. We released the capture antibody through DNA-strand displacement. We coupled the antibodies to the oligonucleotides specified in Table 1 as previously described (Hansen et al. 2017). For immobilization, we used a biotin oligonucleotide immobilized on streptavidin-coated magnetic Dynabeads (Thermo Fisher). The biotinylated oligonucleotide is designed to bind to the oligonucleotide that has been coupled to the capture antibody, and then later (e.g. after washing steps have concluded), using strand displacement, to unbind the capture antibody when the release oligonucleotide is added (Table 1). For detection, we used a PLA to identify when both detection antibodies were bound to the same analyte, with TaqMan probes for qPCR quantification.

FIG. 2 shows the dose response curve of our assay for the S1 portion of the SARS-CoV-2 spike protein added to human saliva. Here we plotted the detected number of counts against the actual number of proteins in the 5 μl sample. The detected number of counts was determined using a calibration curve of the Ct value from a dilution series of an oligonucleotide corresponding to the final, ligated PLA product. Notably, the limit of detection (LOD) was measured to be only 14 molecules from 5 μl of saliva, corresponding to a concentration of 4.7 aM. Here, LOD was determined by extrapolating the concentration which yields a signal equal to background signal plus 3 s.d. of the background signal. Additionally, we obtained a linear response over six orders of magnitude of target protein concentration.

We next used our technique to assay the SARS-COV-2 spike protein in 5 μl saliva samples from 15 COVID+ patients and 24 COVID− patients (FIG. 3). The COVID+ patients were determined to be infected through qPCR on matched nasopharyngeal samples. For catch and release detection, the measured protein count was inferred from the corresponding Ct values obtained from the dose response curve determined in FIG. 2. We also obtained SalivaDirect measurements from the same saliva samples from an authorized lab. SalivaDirect measured the amount of SARS-COV-2 virus through qPCR of virus-specific DNA. For the COVID− patients, 0/24 yielded positive signal above the background for either SalivaDirect or catch and release. For the COVID+ patients, 13/15 yielded positive signal above the background for catch and release, and 11/15 yielded positive signal above the background for SalivaDirect. Notably, 2 samples did not yield a signal in SalivaDirect, but were quantified to have ˜1000 protein molecules in our catch and release assay, demonstrating that we can detect viral protein in samples in which viral nucleic acid was not detected. This is due to the high sensitivity of our technique to detect proteins and also the presence of multiple copies of the spike protein for every viral particle, which only has a single nucleic acid.

In addition to sensitive protein detection, our catch and release assay can be used to measure the protein complex size. We do this by measuring the signal change when adding unlabeled, blocking antibody that competes with the capture antibody in solution. When the target protein complex can bind multiple capture antibodies, the decrease in signal is lower than if each target protein complex can only bind a single capture antibody. The estimated complex size can then be calculated from the ratio of the signal with blocking antibody to signal without blocking antibody and the proportion of unlabeled, blocking antibody, specifically as log (1-signal ratio)/log (blocking antibody proportion). We performed such experiments for both S1 and S2 portions of the SARS-COV-2 spike protein (FIG. 4). Using the same detection antibodies as in FIGS. 2 and 3, we measured the complex size for the S1 portion of the spike-trimer (FIG. 4A). As expected, for a target consisting of the S1 portion of the spike protein, the complex size is ˜1, while for a stabilized version of the ECD of the full spike-trimer, the complex size is ˜3. For both pseudovirus and COVID+ patient samples, the measured complex size is ˜1. This is expected as the native spike-trimer is unstable and the S1 portion detaches from the rest of the virus to form monomers. We also measured complex size using antibodies against the S2 portion of the spike-trimer (FIG. 4B). For the trimer protein we measured an S2 complex size of ˜3 as expected, and for the pseudovirus sample we measured an S2 complex size of ˜57, as each pseudoviral particle contains numerous spike-proteins. This method can potentially be useful in measuring complex size for aggregated proteins, such as those involved in neurodegeneration. This method can be used to measure complex size for any technique that uses an immobilized capture antibody, but our highly sensitive catch and release method allows complex size determination for samples with very few protein particles.

TABLE 1
PLA Oligonucleotide sequences

| | |
|---|---|
| Capture | ACCTAGCTCCACC\3BioTEG\ (SEQ ID NO: 6) |
| Tag | GG\ideoxyU\GGAGC\ideoxyU\AGG\ideoxyU\CAGA\ideoxyU\\ideoxyU\ |
| | A\3AzideN\ (SEQ ID NO: 1) |
| Release | TAA\ideoxyU\C\ideoxyU\GACC\ideoxyU\AGC\ideoxyU\CCACC |
| | (SEQ ID NO: 2) |
| Detection_A | \5AzideN\AAAAACGATTCGAGAACGTGACTGCCATGCAATCATATCTAACCG |
| | GCTGCTATATGATG (SEQ ID NO: 3) |
| Detection_B | \5Phos\ACTATCGTACGCCCGTATGGTAACATTATCGATCGATACCGGACCA |
| | GGTTTCGCAAAAA\3AzideN\ (SEQ ID NO: 4) |
| Splint | G\ideoxyU\ACGA\ideoxyU\AG\ideoxyU\CA\ideoxyU\CA\ideoxyU\A\ |
| | ideoxyU\AG (SEQ ID NO: 5) |
| Block_A | A\ideoxyU\GA\ideoxyU\G |
| Block_B | 5Phos\AC\ideoxyU\A\ideoxyU\C |
| TaqMan Forward | GAACGTGACTGCCATGCAATC (SEQ ID NO: 8) |
| TaqMan Reverse | GAAACCTGGTCCGGTATCGA (SEQ ID NO: 9) |
| TaqMan Probe | FAM-CCGTATGGTAACATTATCG-NFQ (SEQ ID NO: 10) |

### REFERENCES

Fredriksson, S. et al. Protein detection using proximity-dependent DNA ligation assays. Nat Biotechnol 20, 473-477 (2002).
Hansen, C. H., Yang, D., Koussa, M. A., Wong, W. P. Nanoswitch-linked immunosorbent assay (NLISA) for fast, sensitive, and specific protein detection. Proc Natl Acad Sci USA 114, 10367-10372 (2017).
Lundberg, M., Eriksson, A., Tran, B., Assarsson, E. & Fredriksson, S. Homogeneous antibody-based proximity extension assays provide sensitive and specific detection of low-abundant proteins in human blood. Nucleic Acids Res 39, e102 (2011a).
Lundberg. M. et al. Multiplexed homogeneous proximity ligation assays for high-throughput protein biomarker research in serological material. Mol. Cell Proteomics 10, M110 004978 (2011b).
Martell, J. D. et al. A split horseradish peroxidase for the detection of intercellular protein-protein interactions and sensitive visualization of synapses. Nat Biotechnol 34, 774-80 (2016).
Rissin, D. M. et al. Single-molecule enzyme-linked immunosorbent assay detects serum proteins at subfemtomolar concentrations. Nat Biotechnol 28, 595-9 (2010).
Schallmeiner, E. et al. Sensitive protein detection via triple-binder proximity ligation assays. Nat Meth 4, 135-137 (2007).
Vogelstein, B. & Kinzler, K. W. Digital PCR. Proc Natl Acad Sci USA. 96, 9236-41 (1999).
Zhang, D. Y. & Seelig, G. Dynamic DNA nanotechnology using strand-displacement reactions. Nat Chem 3, 103-13 (2011).

### EQUIVALENTS AND SCOPE

All publications, patents, patent applications, publication, and database entries (e.g., sequence database entries) mentioned herein, e.g., in the Background, Summary, Detailed Description, Examples, and/or References sections, are hereby incorporated by reference in their entirety as if each individual publication, patent, patent application, publication, and database entry was specifically and individually incorporated herein by reference. In case of conflict, the present application, including any definitions herein, will control.

Those skilled in the art will recognize, or be able to ascertain, using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one member of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

Where websites are provided, URL addresses are provided as non-browser-executable codes, with periods of the respective web address in parentheses. The actual web addresses do not contain the parentheses.

In addition, it is to be understood that any particular embodiment of the present disclosure may be explicitly excluded from any one or more of the claims. Where ranges are given, any value within the range may explicitly be excluded from any one or more of the claims. Any embodiment, element, feature, application, or aspect of the compositions and/or methods of the disclosure, can be excluded from any one or more claims. For purposes of brevity, all of the embodiments in which one or more elements, features, purposes, or aspects is excluded are not set forth explicitly herein.

What is claimed is:

1. A method of detecting a target analyte, comprising
combining a capture binding partner and a detection binding partner with a sample, under conditions sufficient for the capture binding partner and detection binding partner to bind to a target analyte present in the sample, thereby forming an analyte-binding partner complex
immobilizing the analyte-binding partner complex to a solid support,
removing unbound capture binding partner and detection binding partner,
releasing the analyte-binding partner complex,
transferring the released analyte-binding partner complex to a second container
performing a proximity assay on the released analyte-binding partner complex in solution, thereby detecting the presence of the analyte-binding partner complex.

2. A method of detecting a target analyte, comprising
forming an analyte-binding partner complex comprising a target analyte bound simultaneously to a capture binding partner and two or more detection binding partners, wherein the capture binding partner is immobilized to a solid support before or after binding to the target analyte,
separating the analyte-binding partner complex from unbound capture binding partner and/or unbound detection binding partner,
releasing immobilized analyte-binding partner complex,
transferring the released analyte-binding partner complex to a second container
performing a surface-free proximity assay on the released analyte-binding partner complex, thereby detecting the presence of the analyte-binding partner complex.

3. The method of claim 1 or 2, wherein the target analyte is a protein or a peptide.

4. The method of claim 1 or 2, wherein the capture binding partner is amino acid or nucleic acid in nature.

5. The method of claim 1 or 2, wherein the capture binding partner is an antibody or antigen-binding antibody fragment, a nanobody, a single chain antibody, or an aptamer.

6. The method of claim 1 or 2, wherein the detection binding partner comprises an analyte-specific binding partner conjugated to a detection moiety.

7. The method of claim 6, wherein the analyte-specific binding partner is amino acid or nucleic acid in nature.

8. The method of claim 6, wherein the analyte-specific binding partner is an antibody or antigen-binding antibody fragment, a nanobody, a single chain antibody, or an aptamer.

9. The method of claim 6, wherein the detection moiety is an enzyme substrate, an enzyme, FRET donor or a FRET acceptor.

10. The method of claim 6, wherein the detection moiety is an oligonucleotide.

11. The method of claim 1 or 2, wherein the sample is a bodily sample, optionally a saliva sample, a sputum sample, or a nose or nasopharyngeal swab sample.

12. The method of claim 1 or 2, wherein the target analyte is produced by a pathogen, optionally a virus, a bacterium, or a fungus.

13. The method of claim 1 or 2, wherein the target analyte is SARS-COV-2 spike protein.

14. The method of claim 1 or 2, wherein the target analyte is present in the sample at a concentration of about or at least 5 attoMolar.

15. The method of claim 1 or 2, wherein the solid support is a surface of a well or tube or bead.

16. The method of claim 1 or 2, wherein the capture binding partner is conjugated to a first oligonucleotide, the solid support is conjugated to a second oligonucleotide, the capture binding partner is immobilized to the solid support by hybridization of the first and second oligonucleotides to each other, and wherein the analyte-binding partner complex is released by nucleic acid strand displacement in the presence of a release oligonucleotide, optionally wherein the release oligonucleotide is complementary to the second oligonucleotide.

17. The method of claim 1 or 2, wherein the capture binding partner is immobilized to the solid support by a ultraviolet (UV) cleavable linker, and the analyte-binding partner complex is released by exposure to ultraviolet (UV) light.

18. The method of claim 1 or 2, wherein the capture binding partner is immobilized to the solid support by a nucleic acid linker, and the analyte-binding partner complex is released by contact with a restriction enzyme.

19. The method of claim 1 or 2, wherein during or following the release of the analyte-binding partner complex the solid support is exposed to a quenching agent to prevent re-association of the released analyte-binding partner complex.

20. The method of claim 1 or 2, wherein the detection binding partner is two detection binding partners, and each detection binding partner is conjugated to an oligonucleotide that is a substrate in a proximity assay.

21. The method of claim 1 or 2, wherein the detection binding partner is three detection binding partners, and each detection binding partner is conjugated to an oligonucleotide that is a substrate in a proximity assay.

22. The method of claim 1 or 2, wherein the proximity assay is a proximity ligation assay.

23. The method of claim 1 or 2, wherein the proximity assay is a proximity extension assay.


SEQUENCE LISTING (10 sequences)

SEQ ID NO: 1 - RNA, 20 nt, synthetic construct
  source 1..20: mol_type = other RNA
  modified_base 3: mod_base = OTHER; note = deoxyUridine
  modified_base 9: mod_base = OTHER; note = deoxyUridine
  modified_base 13: mod_base = OTHER; note = deoxyUridine
  modified_base 18..19: mod_base = OTHER; note = deoxyUridine
ggtggagctaggtcagatta

SEQ ID NO: 2 - RNA, 20 nt, synthetic construct
  source 1..20: mol_type = other RNA
  modified_base 4: mod_base = OTHER; note = deoxyUridine
  modified_base 6: mod_base = OTHER; note = deoxyUridine
  modified_base 11: mod_base = OTHER; note = deoxyUridine
  modified_base 15: mod_base = OTHER; note = deoxyUridine
taatctgacctagctccacc

SEQ ID NO: 3 - DNA, 59 bp, synthetic construct
  source 1..59: mol_type = other DNA
aaaaacgattcgagaacgtgactgccatgcaatcatatctaaccggctgctatatgatg

SEQ ID NO: 4 - DNA, 59 bp, synthetic construct
  source 1..59: mol_type = other DNA
actatcgtacgcccgtatggtaacattatcgatcgataccggaccaggtttcgcaaaaa

SEQ ID NO: 5 - RNA, 20 nt, synthetic construct
  source 1..20: mol_type = other RNA
  modified_base 2: mod_base = OTHER; note = deoxyUridine
  modified_base 7: mod_base = OTHER; note = deoxyUridine
  modified_base 10: mod_base = OTHER; note = deoxyUridine
  modified_base 13: mod_base = OTHER; note = deoxyUridine
  modified_base 16: mod_base = OTHER; note = deoxyUridine
  modified_base 18: mod_base = OTHER; note = deoxyUridine
  modified_base 18: mod_base = OTHER; note = deoxyUridine
gtacgatagtcatcatatag

SEQ ID NO: 6 - DNA, 13 bp, synthetic construct
  source 1..13: mol_type = other DNA
acctagctccacc

SEQ ID NO: 7 - skipped

SEQ ID NO: 8 - DNA, 21 bp, synthetic construct
  source 1..21: mol_type = other DNA
gaacgtgactgccatgcaatc

SEQ ID NO: 9 - DNA, 20 bp, synthetic construct
  source 1..20: mol_type = other DNA
gaaacctggtccggtatcga

SEQ ID NO: 10 - DNA, 19 bp, synthetic construct
  source 1..19: mol_type = other DNA
ccgtatggtaacattatcg